(19) United States
(12) Patent Application Publication
(10) Pub. No.: US 2010/0010436 A1
(43) Pub. Date: Jan. 14, 2010

(54) Double burette syringe

(76) Inventors: Jen Chuan Wang, Hacienda Heights, CA (US); Joan Wang, Hacienda Heights, CA (US); Jonathan Wang, Hacienda Heights, CA (US)

Correspondence Address: JEN CHUAN WANG, 2230 Tomich Rd., Hacienda Heights, CA, 91745, US

(21) Appl. No.: 12/217,740

(22) Filed: Jul. 9, 2008

(51) Int. Cl. A61M 31/00

(52) U.S. Cl. 604/89

(57) ABSTRACT

The present invention discloses a device, which comprises a double burette syringe, a stopper, a slidable plate for mixing, and a plurality of mixing bars easily detachable from the mixing plate. This invention relates to devices for storing, delivering, and mixing of a two component double burette syringe.

### CROSS REFERENCE

| | | |
|---|---|---|
| 4040420 | August, 1977 | Speer |
| 4631055 | December 1986 | Redl, et al. |
| 4676406 | June 1987 | Frischmann, et al. |
| 5120305 | June 1992 | Boehringer, et al. |
| 5464396 | November 1995 | Barta el al. |
| 5487606 | January 1996 | Keller |
| 6398761 | June 2002 | Bills, et al. |
| 6620125 | September 2003 | Heinz Redl |
| 6936003 | August 2005 | McIntosh, et al. |
| 7135027 | November, 2006 | Delmotte |

### FIELD OF THE INVENTION

The present invention relates to a storing, delivering and mixing device of a double burette syringe. In particular, the device need no other helping part to mix the two chemicals extruded out from the syringe.

### BACKGROUND OF THE INVENTION

Currently available double burette syringe are useful for storage and dispensing certain gel or viscous liquid. It is especially popular for the two components epoxy adhesive. As the two individual components are squeezed out of the two individual burettes, these two components are directed simultaneously onto any hard surface of his reach and mix thereafter. In fact, because there is no mixing mechanism involved, the two components are not very well mixed resulting in a poor homogeneity. Therefore, one would have to find a separate surface or container and a mixing bar, every time, to mix the two components squeezed out of the double burette syringe, which is additional troublesome. Another prior invention of mixing is having a single burette syringe, with a mixing spiral instrument in the burette, attached right at the outlet of the double burette syringe. Due to the chemical reaction of mixing, the device is clogged. It is obviously a one time use device.

The objects of this invention are (1) to reduce unnecessary waste of the adhesives, and (2) to provide a reusable device used to avoid the trouble on finding the mixing surface/container and a mixing bar when in need and dispose of them afterwards.

### BRIEF SUMMARY OF THE INVENTION

The present invention discloses a device for storing, delivering and mixing of two semi-liquid components without the need for additional parts; the device comprises a double burette syringe, a mixing plate with or without a stopper and one or a plurality of mixing bars easily detachable from the mixing plate. The mixing plate is slidable along the double burette syringe so that it can be extended when the mixing is required and be retracted when the mixing is done.

The mixing device can be a standalone device comprising a plate which can be easily snapped or pushed onto the currently available double burette syringe wherein said double burette syringe is for storage and delivery of two semi-liquid components and the plate can be used for mixing the components extruded out of the double burette syringe. The mixing device can be slidable along the syringe so that a significant portion of the plate is exposed for receiving and mixing the two components extruded out of the double burette syringe. The mixing device may have a protrusion in the rear part so that when the device slides it can be stopped at a suitable position. The mixing device may comprise one or a plurality of mixing bars made by perforation on the side of the plate so that each mixing bar can be easily snapped off from the plate. The mixing device may be made in a plurality of suitable geometrical shape, including a simple bar, thin rod, triangular, spade, and spoon shapes. The mixing device may comprise wall around the edge with suitable height so that less viscous component can be contained and mixed.

### BRIEF DESCRIPTION OF DRAWINGS

FIG. 1 is a perspective view of a double burette syringe and a mixing device in its storing position in accordance with the invention.

FIG. 2 is a perspective view of a double burette syringe and a mixing device, with the mixing bar detached, in its working position in accordance with the invention.

FIG. 3A shows a perspective view of the mixing device with a clamping sleeve of C shaped cross section on a flat mixing plate, a stopper and a mixing bar in accordance with the invention.

FIG. 3B shows a perspective view of the mixing device with a sleeve of circled cross section on a flat mixing plate and a mixing bar in accordance with the invention.

FIG. 4 shows a perspective view of the mixing device with a walled mixing plate and a mixing bar in accordance with the invention.

FIG. 5 shows a perspective view of the mixing device with a curved walled mixing plate and a mixing bar in accordance with the present invention.

### DETAILED DESCRIPTION OF THE INVENTION

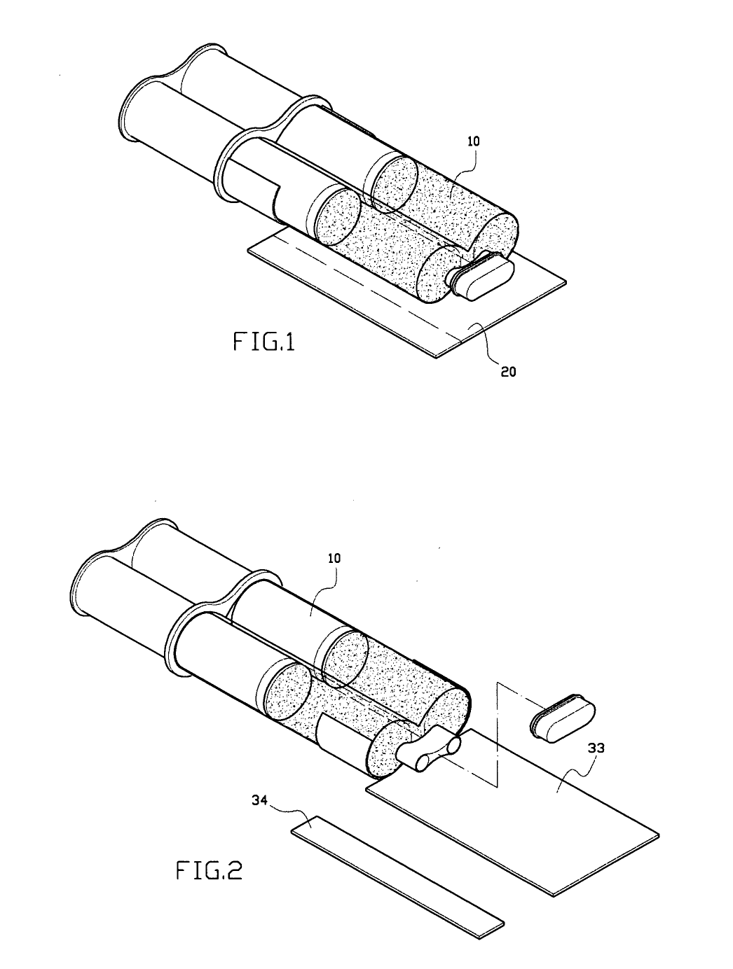

FIG. 1 is a perspective view showing a double burette syringe (10) with the mixing device (20) retracted wherein in FIG. 2, the mixing plate (33) is extended and the mixing bar (34) detached, so that it is ready to mix the semi-liquid extruded out of the double burette syringe.

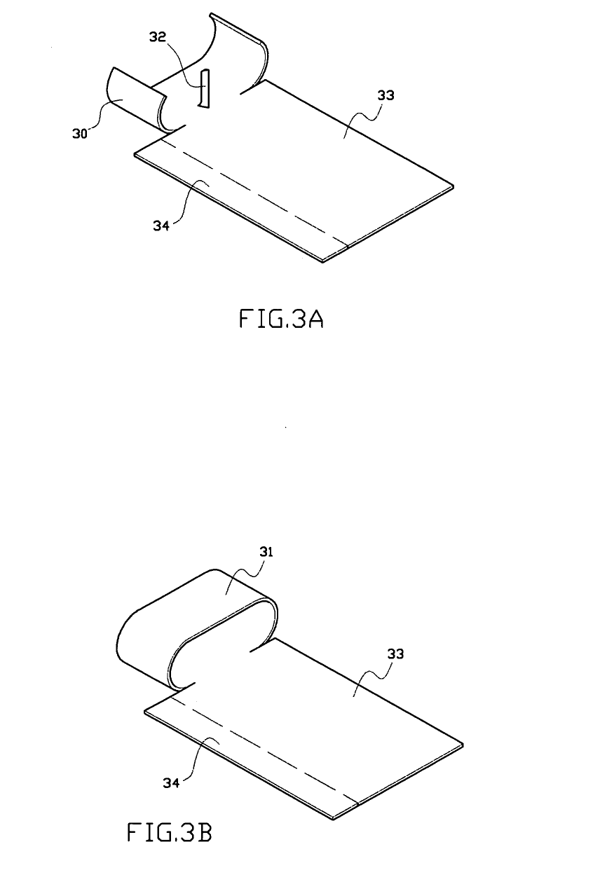

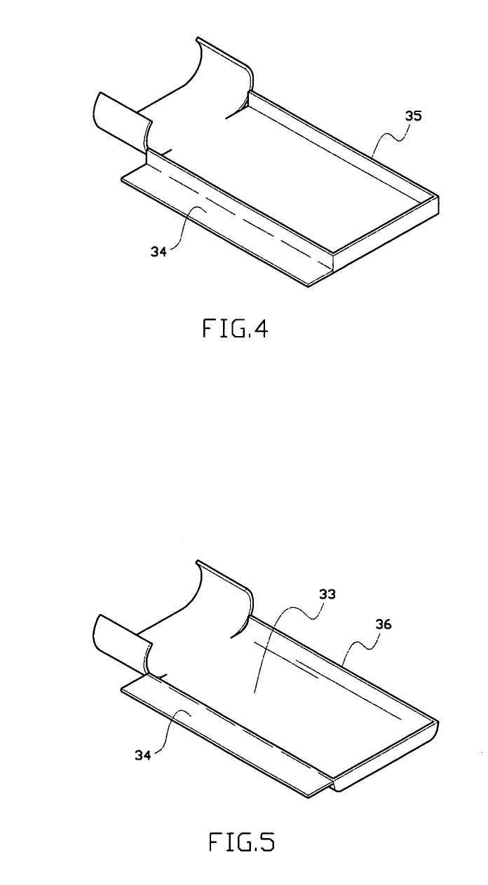

FIG. 3A shows a mixing device with a clamping sleeve (30) having a C shaped cross section in the rear so that it can be slidable along a double burette syringe and a protrusion as a stopper (32) to prevent the device from sliding out off the double burette syringe (10). The open area of the plate (33) is for mixing the semi-liquid and a detachable mixing bar (34) is slightly attached to the plate (33). FIG. 3B shows a mixing device with a sleeve of circled cross section (31). The mixing plate without a stopper can be pushed onto the double syringe through the outlet end. FIG. 4 shows a mixing device with a mixing bar (34) slightly attached on the mixing plate. The plate (33) has a wall (35) with suitable height along the edges of the plate. FIG. 5 shows a mixing device having a curved wall (36) and a mixing bar (34) slightly attached on the mixing plate (33).

In conclusion, the present invention discloses a device for delivering and mixing two semi-liquid components without the need for additional parts. Also disclosed in the present invention is a mixing device comprising a plate which can be easily snapped or pushed onto the currently available double burette syringe and the plate can be used for mixing the two components extruded out of the double burette syringe. To the best of our knowledge, this device has not been disclosed by any other parties.

Although the description above contains many specificities, these should not be construed as limiting the scope of the invention but as merely providing the illustrations of some of the presently preferred embodiments of this invention. Thus the scope of this invention should be determined by the appended claims and their legal equivalents, rather than by the examples given.

1. A device for storing, delivering and mixing two semi-liquid components without the need for additional parts.

2. A device according to claim 1 comprising a double burette syringe, a mixing plate with or without a stopper and one or a plurality of mixing bars easily detachable from the mixing plate.

3. A device according to claim 2 wherein the plate is slidable along the double burette syringe so that it can be extended when the mixing is required and be retracted when the mixing is done.

4. A mixing device comprising a plate which can be easily snapped or pushed onto the currently available double burette syringe wherein said double burette syringe is for storage and delivery of two semi-liquid components and the plate can be used for mixing the components extruded out of the double burette syringe.

5. The mixing device according to claim 4 wherein the plate, once snapped or pushed onto the double burette syringe, is slidable along the syringe so that a significant portion of the plate is exposed for receiving and mixing the two components extruded out of the double burette syringe.

6. The mixing device according to claim 5 wherein there is a protrusion in the rear part so that when the device slides along the syringe, it can be stopped at a suitable position.

7. The mixing device according to claim 4 comprising one or a plurality of mixing bars made by perforation on side of the plate so that the each mixing bar can be easily snapped off from the plate.

8. The mixing device according to claim 7 wherein said mixing bar can be made in a plurality of suitable geometrical shape, including a simple bar, thin rod, triangular, spade, spoon and beveled shapes.

9. The mixing device according to claim 4 wherein the plate has wall around the edge with suitable height so that less viscous component can be contained and mixed.
